(19) United States
(12) Patent Application Publication
(10) Pub. No.: US 2024/0252117 A1
(43) Pub. Date: Aug. 1, 2024

(54) MAGNETIC RESONANCE IMAGING APPARATUS AND TABLE APPARATUS

(71) Applicant: CANON MEDICAL SYSTEMS CORPORATION, Otawara-shi (JP)

(72) Inventors: Keisuke OKUZUMI, Utsunomiya (JP); Kaoru IKEDA, Nasushiobara (JP); Naoko YAMAGUCHI, Utsunomiya (JP); Shoji ISHIZAKI, Nasushiobara (JP)

(73) Assignee: CANON MEDICAL SYSTEMS CORPORATION, Otawara-shi (JP)

(21) Appl. No.: 18/405,101

(22) Filed: Jan. 5, 2024

(30) Foreign Application Priority Data
Jan. 27, 2023 (JP) 2023-010952

(51) Int. Cl. A61B 5/00; A61B 5/055

(52) U.S. Cl. CPC A61B 5/704; A61B 5/055

(57) ABSTRACT

A magnetic resonance imaging apparatus according to an embodiment includes a table apparatus and a processor. The table apparatus includes a table-top on which a subject is placed. The processor acquires subject position information indicating a position of the subject on the table-top. The processor acquires weight information indicating a weight of the subject. The processor calculates an amount of deflection occurred in the table-top based on the subject position information and the weight information. The processor generates, based on the amount of deflection of the table-top, first path information indicating a path along which the table-top is moved from the table apparatus to a gantry where imaging of the subject is performed. The processor operates a moving mechanism capable of moving the table-top from the table apparatus to the gantry to move the table-top from the table apparatus to the gantry along the path indicated by the first path information.

### CROSS-REFERENCE TO RELATED APPLICATIONS

This application is based upon and claims the benefit of priority from Japanese Patent Application No. 2023-010952, filed on Jan. 27, 2023; the entire contents of which are incorporated herein by reference.

### FIELD

Embodiments disclosed in the present specification and drawings relate to a magnetic resonance imaging apparatus and a table apparatus.

### BACKGROUND

With conventional magnetic resonance imaging (MRI) apparatuses, a subject is moved into a gantry by moving a table-top on which the subject is placed into the gantry. Usage of magnetic materials or carbon fibers is not allowed for the table-top of the MRI apparatus, which makes a cantilevered table-top difficult to implement in relation to strength. Therefore, in magnetic resonance imaging, a member for supporting the table-top (e.g., rail structure) is usually provided on a gantry side.

Moreover, a table that supports the table-top outside the gantry is disposed with a gap between the table and the gantry; therefore, gaps may be generated between the table-top and the gantry extending in a horizontal direction and a height direction. There have been proposed technologies to move the table, which supports the table-top, up to the same level as a table-top supporting member provided on the gantry and then move the table-top in the horizontal direction.

### BRIEF DESCRIPTION OF THE DRAWINGS

FIG. 1 is a block diagram illustrating an example of the configuration of a magnetic resonance imaging apparatus according to a first embodiment;

FIG. 2 is a schematic diagram illustrating an example of positional relationship between a moving table and a gantry according to the first embodiment;

FIG. 3 is a flowchart illustrating an example of a process performed by the magnetic resonance imaging apparatus according to the first embodiment;

FIG. 4 is a block diagram illustrating an example of the configuration of a magnetic resonance imaging apparatus according to a second embodiment;

FIG. 5 is a flowchart illustrating an example of a process performed by the magnetic resonance imaging apparatus according to the second embodiment;

FIG. 6 is a block diagram illustrating an example of the configuration of a magnetic resonance imaging apparatus according to a third embodiment;

FIG. 7 is a flowchart illustrating an example of a process performed by the magnetic resonance imaging apparatus according to the third embodiment;

FIG. 8 is a block diagram illustrating an example of the configuration of a magnetic resonance imaging apparatus according to a fourth embodiment; and

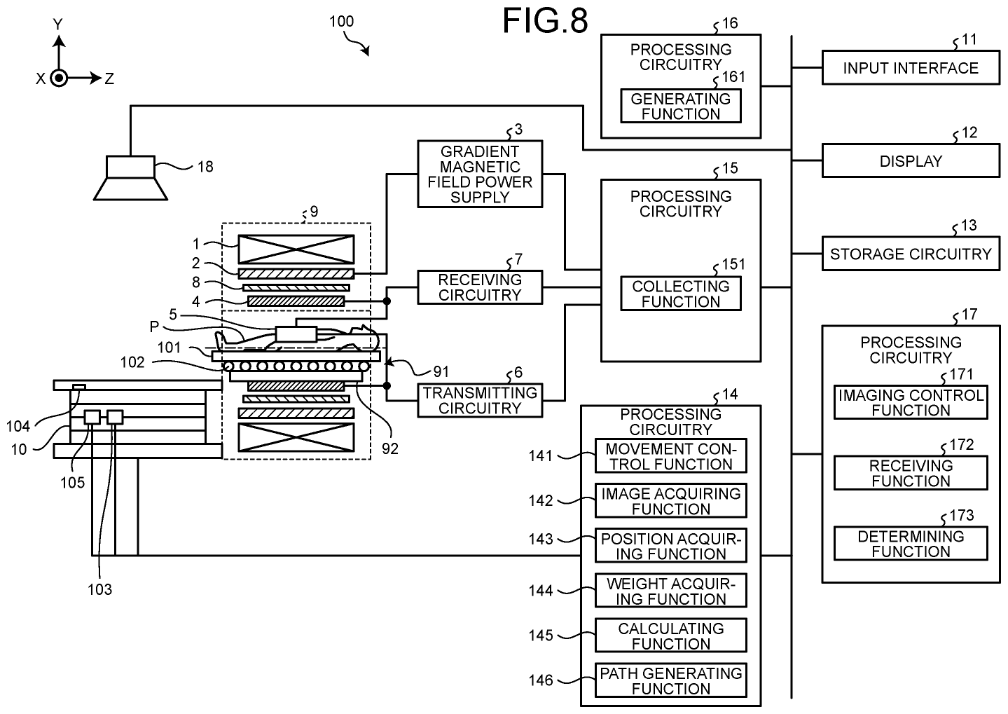

FIG. 9 is a block diagram illustrating an example of the configuration of a magnetic resonance imaging apparatus according to a fifth embodiment.

### DETAILED DESCRIPTION

A magnetic resonance imaging apparatus according to an embodiment includes a table apparatus and a processor. The table apparatus includes a table-top on which a subject is placed. The processor acquires subject position information indicating a position of the subject on the table-top. The processor acquires weight information indicating a weight of the subject. The processor calculates an amount of deflection occurred in the table-top based on the subject position information and the weight information. The processor generates, based on the amount of deflection of the table-top, first path information indicating a path along which the table-top is moved from the table apparatus to a gantry where imaging of the subject is performed. The processor operate a moving mechanism capable of moving the table-top from the table apparatus to the gantry to move the table-top from the table apparatus to the gantry along the path indicated by the first path information.

Explained in detail below are embodiments of a magnetic resonance imaging apparatus and a table apparatus with reference to the accompanied drawings.

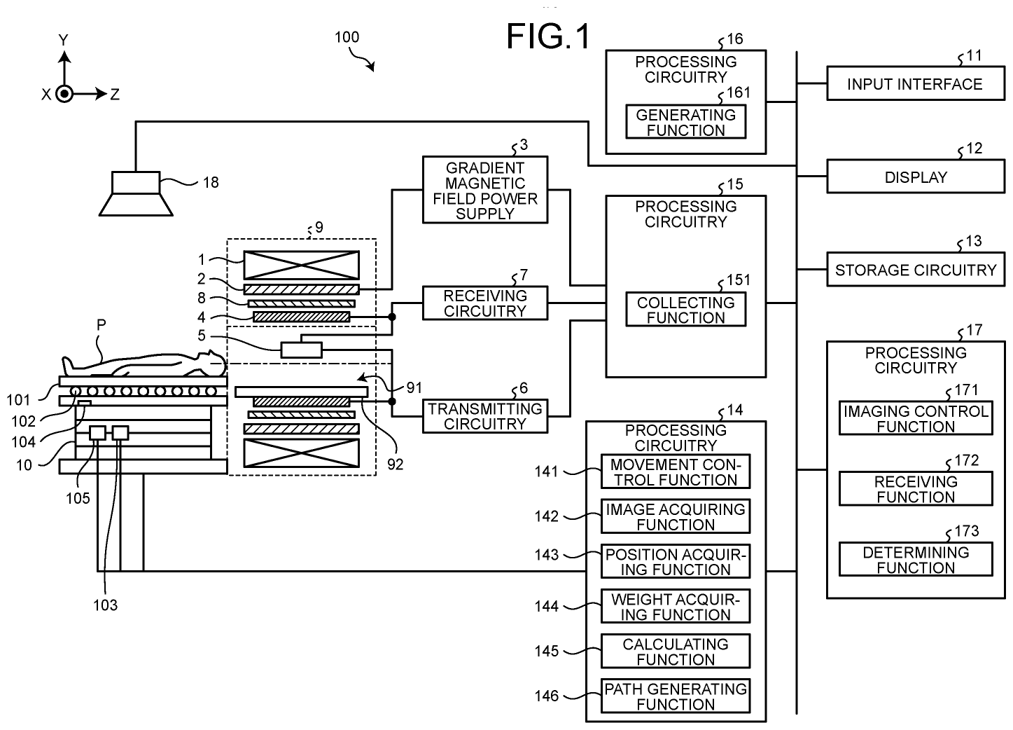

FIG. 1 is a block diagram illustrating an example of the configuration of a magnetic resonance imaging apparatus according to a first embodiment.

For example, as illustrated in FIG. 1, a magnetic resonance imaging apparatus 100 includes a static magnetic field magnet 1, a gradient magnetic field coil 2, a gradient magnetic field power supply 3, a whole-body RF coil 4, a local RF coil 5, transmitting circuitry 6, receiving circuitry 7, an RF shield 8, a gantry 9, a table 10, an input interface 11, a display 12, storage circuitry 13, processing circuitry 14 to 17, and a camera 18.

The static magnetic field magnet 1 generates a static magnetic field in an imaging space where a subject P is placed. Specifically, the static magnetic field magnet 1 is formed in a hollow, substantially-cylindrical shape (including a shape whose cross-section perpendicular to its central axis is elliptical). The static magnetic field magnet 1 generates a static magnetic field in an imaging space, which is formed circumferentially inside itself. For example, the static magnetic field magnet 1 is a superconducting magnet, a permanent magnet, etc. The superconducting magnet herein, for example, includes a container filled with a coolant, such as liquid helium, and a superconducting coil immersed in the container.

The gradient magnetic field coil 2 is located inside the static magnetic field magnet 1 and generates a gradient magnetic field in the imaging space where the subject P is placed. Specifically, the gradient magnetic field coil 2 is formed in a hollow, substantially-cylindrical shape (including a shape whose cross-section perpendicular to the central axis is elliptical). The gradient magnetic field coil 2 has X, Y, and Z coils corresponding to mutually orthogonal X, Y, and Z axes, respectively.

The X, Y, and Z coils generate gradient magnetic fields that vary linearly along the respective axis directions in the imaging space based on current supplied from the gradient magnetic field power supply 3. Herein, the Z axis is set to extend along a magnetic flux of the static magnetic field generated by the static magnetic field magnet 1. Moreover, the X axis is set to extend along a horizontal direction orthogonal to the Z axis, and the Y axis is set to extend along a vertical direction orthogonal to the Z axis. Herein, the X, Y, and Z axes form a device coordinate system unique to the magnetic resonance imaging apparatus 100.

The gradient magnetic field power supply 3 supplies a current to the gradient magnetic field coil 2 to generate a gradient magnetic field in the imaging space. Specifically, the gradient magnetic field power supply 3 supplies currents to the X, Y, and Z coils of the gradient magnetic field coil 2 individually to generate in the imaging space gradient magnetic fields that vary linearly along a readout direction, a phase encoding direction, and a slice direction, respectively, those directions being orthogonal to each other.

Herein, an axis along the readout direction, an axis along the phase encoding direction, and an axis along the slice direction form a logical coordinate system for defining a slice or volume region to be imaged.

Specifically, a gradient magnetic field along each of the readout direction, the phase encoding direction, and the slice direction is superimposed on the static magnetic field generated by the static magnetic field magnet 1 to impart spatial position information to a nuclear magnetic resonance (NMR) signal generated from the subject P. Specifically, a gradient magnetic field in the readout direction imparts readout-directional position information to the NMR signal by changing a frequency of the NMR signal according to a position in the readout direction.

Moreover, a gradient magnetic field in the phase-encoding direction imparts phase-encoding-directional position information to the NMR signal by changing a phase of the NMR signal according to a position in the phase-encoding direction. Furthermore, a gradient magnetic field in the slice direction imparts slice-directional position information to the NMR signal.

For example, a gradient magnetic field in the slice direction is used to determine direction, thickness, and number of slices when the imaging area is a slice area (2D imaging). The gradient magnetic field in the slice direction is used to change a phase of the NMR signal according to a position in the slice direction when the imaging area is a volume area (3D imaging).

The whole-body RF coil 4 is located circumferentially inside the gradient magnetic field coil 2. The whole-body RF coil 4 transmits an RF pulse to the subject P who is placed in the imaging space and receives an NMR signal generated from the subject P under the influence of the RF pulse.

Specifically, the whole-body RF coil 4 is formed in a hollow, substantially-cylindrical shape (including a shape whose cross-section perpendicular to the central axis is elliptical). The whole-body RF coil 4 applies an RF magnetic field to the subject P who is in the imaging space, which lies circumferentially inside itself, based on the RF pulse supplied from the transmitting circuitry 6. The whole-body RF coil 4 receives the NMR signal generated from the subject P by the influence of the RF magnetic field and outputs the received NMR signal to the receiving circuitry 7.

For example, the whole-body RF coil 4 is a birdcage coil or a transverse electromagnetic (TEM) coil. Note that the whole-body RF coil 4 does not necessarily have both transmitting and receiving functions. The whole-body RF coil 4 may have only the transmitting function.

The local RF coil 5 is placed near the subject P during imaging. The local RF coil 5 transmits an RF pulse to the subject P who is in the imaging space and receives an NMR signal generated from the subject P under the influence of the RF pulse.

Specifically, the local RF coil 5 is prepared for each site of the subject P and arranged near the site to be imaged when imaging of the subject P is being performed. The local RF coil 5 applies an RF magnetic field to the subject P based on an RF pulse supplied from the transmitting circuitry 6.

The local RF coil 5 then receives an NMR signal generated from the subject P by the influence of the RF magnetic field and outputs the received NMR signal to the receiving circuitry 7. For example, the local RF coil 5 is a surface coil or a phased array coil that is formed of a combination of two or more surface coils as coil elements. Note that the local RF coil 5 does not necessarily have both transmitting and receiving functions. The local RF coil 5 may have only the receiving function.

The transmitting circuitry 6 outputs an RF pulse corresponding to a resonance frequency (Larmor frequency) specific to a target nucleus in the static magnetic field to the whole-body RF coil 4 or the local RF coil 5.

The receiving circuitry 7 generates NMR data based on the NMR signal output from the whole-body RF coil 4 or the local RF coil 5 and outputs the generated NMR data to the processing circuitry 15. Moreover, the receiving circuitry 7 includes a plurality of analog to digital converters (ADCs). The ADCs convert the NMR signal output from the whole-body RF coil 4 or the local RF coil 5 into digital data. The ADCs are an example of a conversion unit. The ADCs will be described later.

The RF shield 8 is placed between the gradient magnetic field coil 2 and the whole-body RF coil 4 to shield the gradient magnetic field coil 2 from the RF magnetic field generated by the whole-body RF coil 4. Specifically, the RF shield 8 is formed in a hollow, substantially-cylindrical shape (including a shape whose cross-section perpendicular to the central axis of the cylinder is elliptical). The RF shield 8 is located in the space lying circumferentially inside the gradient magnetic field coil 2 to cover an outer circumference of the whole-body RF coil 4.

The gantry 9 has a hollow bore 91 formed in a substantially-cylindrical shape (including a shape whose cross-section perpendicular to the central axis is elliptical). The gantry 9 accommodates therein the static magnetic field magnet 1, the gradient magnetic field coil 2, the whole-body RF coil 4, the RF shield 8, and a gantry rail 92.

Specifically, the gantry 9 accommodates therein each of the above-mentioned members such that the whole-body RF coil 4 is arranged circumferentially outside the bore 91, the RF shield 8 is arranged circumferentially outside the whole-body RF coil 4, the gradient magnetic field coil 2 is arranged circumferentially outside the RF shield 8, and the static magnetic field magnet 1 is arranged circumferentially outside the gradient magnetic field coil 2.

Moreover, the gantry 9 has the gantry rail 92 that supports a table-top 101, on which the subject P is placed, such that the table-top 101 is movable in the bore 91. The gantry rail 92 is provided, for example, in a lower part within the bore 91 of the gantry 9, extending along an axial direction of the bore 91. Herein, a space inside the bore 91 of the gantry 9 operates as the imaging space in which the subject P is placed during imaging.

The table 10 includes the table-top 101 on which the subject P is placed, a moving member 102 that moves the table-top 101 to the gantry 9, a driving member 103 that changes height of the table 10, a table-top position detection sensor 104 that detects position of the table-top 101, and a table height detection sensor 105 that detects height of the table 10. The table 10 is an example of a table apparatus. The table 10 moves the table-top 101 on which the subject P is placed into the bore 91 (imaging space) of the gantry 9 when imaging of the subject P is performed. For example, the table 10 is set so that a longitudinal direction (Z-axis direction) of the table-top 101 is parallel to the central axis of the static magnetic field magnet 1. Note that, in an initial state before the table-top 101 is moved into the gantry 9, there is a gap between the table 10 and the gantry 9.

The moving member 102 is a member for moving the table-top 101 into the imaging space in the gantry 9, and is provided a lower part of the table-top 101. The moving member 102 is, for example, a roller for moving the table-top 101 in the Z-axis direction. Note that a configuration is explained in the present embodiment in which the moving member 102 is attached to the table-top 101. The moving member 102 may be attached to an upper surface of the table 10 that supports the table-top 101 from below.

The driving member 103 is a member for moving height of the table 10 in the up-and-down direction (Y-axis direction) and is provided inside the table 10. The driving member 103 is also used to move the table-top 101 in the Z-axis direction. The driving member 103 is, for example, a driving motor. The moving member 102 and the driving member 103 are examples of moving mechanisms. The moving mechanisms can move the table-top 101 in the horizontal direction and the vertical direction.

The table-top position detection sensor 104 is a detection sensor that detects position of the table-top 101 in the Z-axis direction (hereinafter, also referred to as “table-top position”). The table-top position detection sensor 104 is provided under the table-top 101, for example. Specifically, the table-top position detection sensor 104 detects a Z-axis-directional position of the table-top 101 moving on the table 10.

Note that the table-top position detected by the table-top position detection sensor 104 may be a relative Z-axial-directional position of the table-top 101 relative to the table 10. For example, the table-top position detection sensor 104 uses a position of the table-top 101 in the initial state before the table-top 101 is moved into the gantry 9 as a reference position and detects a distance by which the table-top 101 has moved in the Z-axis direction from the reference position as the table-top position. Moreover, the table-top position detection sensor 104 transmits the detected table-top position to the processing circuitry 14.

The table height detection sensor 105 is a detection sensor that detects height of the table 10 in the Y-axis direction. The table height detection sensor 105 is, for example, provided inside the table 10. Specifically, the table height detection sensor 105 detects Y-axis-directional position (height) of the table 10 or the table-top 101 moved by the driving member 103. Moreover, the table height detection sensor 105 transmits the detected height to the processing circuitry 14.

The input interface 11 receives input operations of various instructions and various information from an operator. Specifically, the input interface 11 is connected to the processing circuitry 17. The input interface 11 converts an input operation received from the operator into an electric signal and outputs the electric signal to the processing circuitry 17.

For example, the input interface 11 is implemented by components, such as a trackball, a switch button, a mouse, and a keyboard, with which settings, etc., are made for an imaging condition and a region of interest (ROI); a touchpad on which an input operation is performed with a touch onto its operation screen; a touchscreen that is an integration of a display screen and a touchpad; non-contact input circuitry using an optical sensor; voice input circuitry, and the like.

Note that, in the present specification, the input interface 11 is not limited to ones having physical operation components such as a mouse and a keyboard. For example, examples of the input interface 11 include electric-signal processing circuitry that receives an electric signal corresponding to an input operation from an external input device, which is provided separately from the apparatus, and outputs the electric signal to the control circuitry.

The display 12 displays various types of information thereon. Specifically, the display 12 is connected to the processing circuitry 17. The display 12 converts data of various types of information received from the processing circuitry 17 into an electric signal for display and outputs the electric signal. For example, the display 12 is implemented by a liquid crystal monitor, a CRT monitor, a touch panel, etc.

The camera 18 is formed with a charge coupled device (CCD) image sensor or a complementary metal oxide semiconductor (CMOS) image sensor. The camera 18 captures an image of the subject P placed on the table-top 101. The camera 18 generates an optical image of the subject P (hereinafter referred to as “first captured image”) and transmits the first captured image to the processing circuitry 14.

Note that the camera 18 is preferably provided at a position able to image an upper surface of the table-top 101 before the table-top 101 is inserted into the bore 91. For example, the camera 18 is provided on an outer wall surface of the gantry 9 closer to the table 10 or on a ceiling, wall surface, etc., of a room where the magnetic resonance imaging apparatus 100 is installed.

The storage circuitry 13 stores therein various types of data. Specifically, the storage circuitry 13 is connected to the processing circuitry 14 to 17. The storage circuitry 13 stores therein various types of data input and output by the respective processing circuitry. For example, the storage circuitry 13 is implemented by a semiconductor memory element, such as a random access memory (RAM) and a flash memory, a hard disk, an optical disk, etc.

The processing circuitry 15 has a collecting function 151. The collecting function 151 collects k-space data by executing various pulse sequences. Specifically, the collecting function 151 executes various pulse sequences by driving the gradient magnetic field power supply 3, the transmitting circuitry 6, and the receiving circuitry 7 according to sequence execution data output from the processing circuitry 17.

Herein, the sequence execution data is data that represents a pulse sequence, and is information that defines timing and strength of a current supplied by the gradient magnetic field power supply 3 to the gradient magnetic field coil 2, timing and strength of an RF pulse supplied by the transmitting circuitry 6 to the whole-body RF coil 4, timing at which the receiving circuitry 7 performs sampling of NMR signals, etc.

The collecting function 151 receives NMR data output from the receiving circuitry 7 as a result of the pulse sequence execution and stores the NMR data in the storage circuitry 13. The NMR data stored in the storage circuitry 13 is given position information along each of the readout direction, the phase encoding direction, and the slice direction by each of the above-mentioned gradient magnetic fields such that the NMR data is stored as k-space data representing a two- or three-dimensional k-space.

The processing circuitry 16 has a generating function 161. The generating function 161 generates an image from the k-space data collected by the processing circuitry 15. Specifically, the generating function 161 reads the k-space data collected by the processing circuitry 15 from the storage circuitry 13 and performs reconstruction processing, such as Fourier transformation, on the read k-space data to generate a two- or three-dimensional image. The generating function 161 then stores the generated image in the storage circuitry 13.

The processing circuitry 17 has an imaging control function 171, a receiving function 172, and a determining function 173. The imaging control function 171 performs various types of imaging by controlling various components of the magnetic resonance imaging apparatus 100. Specifically, the imaging control function 171 displays a graphical user interface (GUI) on the display 12 to receive input operations of various instructions and various information from the operator and controls various components of the magnetic resonance imaging apparatus 100 according to the input operations received via the input interface 11.

For example, the imaging control function 171 generates sequence execution data based on an imaging condition input by the operator and outputs the generated sequence execution data to the processing circuitry 15, so that k-space data is collected. Moreover, for example, the imaging control function 171 controls the processing circuitry 16, so that an image is reconstructed from the k-space data collected by the processing circuitry 15. Furthermore, for example, the imaging control function 171 reads an image from the storage circuitry 13 in response to a request from the operator and displays the read image on the display 12.

The receiving function 172 receives an input from a user. For example, the receiving function 172 receives, via the input interface 11, an input from the user of weight information indicating weight of the subject P (for example, body weight of the subject P, etc.) and information for setting an imaging condition or a region of interest. Moreover, for example, the receiving function 172 receives, via the input interface 11, an input of posture of the subject P placed on the table-top 101 or information with which the posture can be identified.

Herein, the posture of subject P means, for example, head first, in which the subject P is placed with the head pointing toward the gantry 9, feet first, in which the subject P is placed with the feet pointing toward the gantry 9, etc.

The determining function 173 determines an imaging condition for the subject P. For example, the determining function 173 determines an imaging condition for the subject P including a site of the subject P to be imaged based on the information for setting an imaging condition or a region of interest received by the receiving function 172. Note that the determining function 173 may determine an imaging condition based on test order information, etc., managed by a hospital information system (HIS) or the like.

The processing circuitry 14 has a movement control function 141. The movement control function 141 is an example of a movement control unit. The movement control function 141 controls operation of the table 10 by outputting an electric signal for control to the table 10.

For example, the movement control function 141 receives, via the input interface 11, an instruction from the operator to move the table-top 101 in the longitudinal direction, the up-and-down direction, or the left-and-right direction, and operates a moving mechanism (the moving member 102 and the driving member 103) of the table 10 so that the table-top 101 is moved according to the received instruction. Note that the processing circuitry 14 may be included in the table 10.

By the way, it has been known that the table 10 and the table-top 101 may deflect due to the load of the subject P. Moreover, an amount of deflection varies depending on where the subject P is placed on the table-top, how the subject P is placed (head first or feet first), etc.

Therefore, for example, even if the height of the table 10 is raised to the same level as the table-top supporting member provided on the gantry 9, the deflection occurred in the table 10 and the table-top 101 may cause rattling (shaking or vibration) when the table-top 101 is moved from the table 10 to the gantry 9.

Such rattling in the table-top 101 during movement of the table-top may awake the subject P from sedation (for example, drug-induced somnolence) or make the subject P uncomfortable.

To solve the problem, the processing circuitry 14 includes, in addition to the movement control function 141, an image acquiring function 142, a position acquiring function 143, a weight acquiring function 144, a calculating function 145, and a path generating function 146. The movement control function 141 operates the moving mechanism capable of moving the table-top 101 from the table 10 to the gantry 9 to move the table-top 101 to the gantry 9 along a path indicated by first path information. Note that the first path information generated by the path generating function 146 will be described later.

The image acquiring function 142 acquires the first captured image. The image acquiring function 142 is an example of an image acquiring unit. Specifically, the image acquiring function 142 acquires the first captured image that is an image of the upper surface of the table-top 101 captured by the camera 18, the image including the subject P placed on the table-top 101.

The position acquiring function 143 acquires subject position information indicating position of the subject P placed on the table-top 101. The position acquiring function 143 is an example of a position acquiring unit. Specifically, the position acquiring function 143 detects a position of the subject P on the table-top 101 based on the first captured image that is an image of the upper surface of the table-top 101 acquired by the image acquiring function 142, thereby acquiring the subject position information indicating the position.

Moreover, the position acquiring function 143 may detect a posture of the subject P on the table-top 101 from the first captured image that is acquired by the image acquiring function 142, thereby acquiring the posture as the subject position information. Note that the position acquiring function 143 may acquire a posture of the subject P received by the receiving function 172 as the subject position information.

The weight acquiring function 144 acquires weight information indicating weight of the subject P. The weight acquiring function 144 is an example of a weight acquiring unit. Specifically, the weight acquiring function 144 acquires weight information indicating a weight of the subject P received by the receiving function 172.

The calculating function 145 calculates an amount of deflection that occurs in the table-top 101. The calculating function 145 is an example of a calculating unit. Specifically, the calculating function 145 develops a load on the table-top 101 generated by the subject P placed based on the subject position information acquired by the position acquiring function 143 and the weight information acquired by the weight acquiring function 144 and calculates an amount of deflection of the table-top 101 in the Y-axis direction.

More specifically, the calculating function 145 calculates an amount of deflection in the Y-axis direction at a Z-axis-directional end of the table-top 101 based on the subject position information and the weight information. Herein, the Z-axis-directional end of the table-top 101 means a leading end of the table-top 101 when it is moved in the Z-axis direction. Note that any methods including well-known methods can be used to calculate the amount of deflection. The calculating function 145 may calculate the amount of deflection using specification information indicating rigidity or structure of the table-top 101 and the table 10.

Moreover, along with the amount of deflection occurred in the table-top 101, the calculating function 145 may calculate an amount of deflection occurred in the upper surface of the table 10 that supports the table-top 101 from below. In this case, similar to the table-top 101, the calculating function 145 calculates an amount of deflection in the Y-axis direction at the Z-axis-directional end of the table 10.

The path generating function 146 generates first path information that indicates a path along which the table-top 101 is moved from the table 10 to the gantry 9 where imaging of the subject P is performed. The path generating function 146 is an example of a path generating unit.

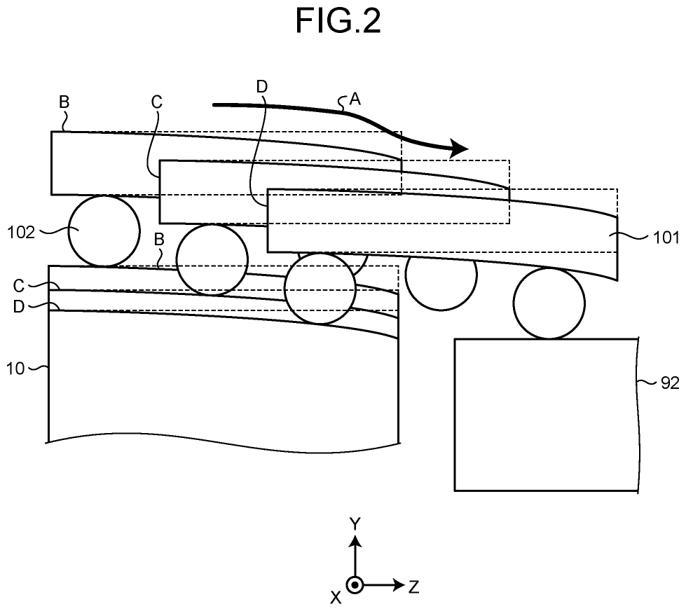

Explained below with reference to FIG. 2 is a path along which the table-top 101 is moved from the table 10 to the gantry 9. FIG. 2 is a schematic diagram illustrating an example of a path of the table-top 101 according to the first embodiment. FIG. 2 illustrates a path A indicating a path along which the table-top 101 moves and deflection states B, C, and D, each indicating a state of the table-top 101 and the table 10 deflecting in the negative Y-axis direction.

The path generating function 146 generates first path information indicating a path along which the table-top 101 is moved from the table 10 to the gantry 9 based on an amount of deflection of the table-top 101 calculated by the calculating function 145. The first path information includes at least a vertical height of the table-top 101 at each position through which the table-top 101 is moved from the table 10 to the gantry 9. For example, the first path information includes a height of the table 10 before the table-top 101 is moved to the gantry 9, a height of the table 10 immediately after the table-top 101 moved to the gantry 9, and a height of the table 10 after the table-top 101 moved to the gantry 9.

Herein, height of the table 10 is set based on an amount of deflection calculated by the calculating function 145 at each position in a process of moving the table-top 101. Specifically, the path generating function 146 sets the height with the amount of deflection of the table-top 101 being taken into consideration so that the moving member 102 attached to the table-top 101 cannot ride on the gantry rail 92 of the gantry 9 during the process of moving the table-top 101 from the table 10 to the gantry 9. Note that it is assumed that the height of the gantry 9 (and the gantry rail 92) has already been known.

For example, as a path before movement of the table-top 101 in the Z-axis direction, the path generating function 146 generates first start-up information to instruct movement of the table 10 to a position higher than the gantry rail 92 of the gantry 9 based on an amount of deflection at the end of the table-top 101 (and the table 10) (the deflection state B illustrated in FIG. 2).

Note that, from the viewpoint of improvement in throughput, the height of the table 10 increases to a level high enough to prevent the moving member 102 of the table-top 101 from riding on the gantry rail 92 of the gantry 9 when the table-top 101 is moving in the Z-axis direction. Moreover, the height of the table 10 may be adjusted in accordance with the weight of the subject P.

Furthermore, as a path after the table-top 101 starts moving in the Z-axis direction, the path generating function 146 generates first start-up information to instruct decrease (movement) in height of the table 10 to the height of the gantry rail 92 based on an amount of deflection at the end of the table-top 101 (and the table 10) (the deflection state C illustrated in FIG. 2) so that the moving member 102 of the table-top 101 rides on the gantry rail 92 of the gantry 9.

Furthermore, the path generating function 146 generates, as a path after the table-top 101 starts moving in the Z-axis direction and lands on the gantry 9, first start-up information to instruct to make the height of the table 10 and the height of the gantry rail 92 equal based on an amount of deflection at the end of the table-top 101 (and the table 10) (the deflection state D illustrated in FIG. 2) so that the moving member 102 of the table-top 101 rides on the gantry rail 92 of the gantry 9. Note that it is preferable that the height of the table 10 decreases (moves down) gradually so that the moving member 102 of the table-top 101 softly lands on the gantry rail 92.

Moreover, the path generating function 146 may generate first path information that includes speed of, timing to accelerate, and timing to stop the table-top 101 moving in the Z-axis direction.

The movement control function 141 adjusts, in the process of moving the table-top 101 into the gantry 9, the height of the table 10 by controlling the driving member 103 based on the first path information. As a result, the table-top 101 softly lands on the gantry rail 92 while following the path A illustrated in FIG. 2.

When speed, etc. of the table-top 101 is included in the first path information, the movement control function 141 moves the table-top 101 into the gantry 9 by controlling the moving member 102 and the driving member 103 based on the first path information (so that the table-top 101 is moved in the Z-axis direction). In this case, the table-top 101 softly lands on the gantry rail 92, while following the path A illustrated in FIG. 2, as well.

The table-top 101 moves to the gantry rail 92 under control of the movement control function 141 based on the first path information generated by the path generating function 146, while the height of the table 10 (in the Y-axis direction) interlocks with the moving position of the table-top 101 (in the Z-axis direction).

Herein, the above-mentioned processing circuitry 14 to 17 is implemented, for example, by a processor. In this case, the processing functionality of each processing circuitry is stored in the storage circuitry 13, for example, in the form of a program executable by a computer. Then, each processing circuitry reads each program from the storage circuitry 13 and executes the program to realize the processing functionality corresponding to the program. In other words, when reading the respective programs, each processing circuitry is given its functionality as illustrated in FIG. 1.

Although it has been explained herein that each processing circuitry is implemented by a single processor, embodiments are not limited thereto. It is allowable to configure each processing circuitry by a combination of two or more independent processors and realize each processing function when each processor executes a program. In addition, the processing functions of each processing circuitry may be distributed among two or more processing circuits or integrated into one processing circuit as appropriate for realization.

Moreover, although it has been explained in the example illustrated in FIG. 1 that the single storage circuitry 13 stores the program corresponding to each processing function, such configuration is allowable in which two or more storage circuits are arranged in a distributed manner and the processing circuitry reads corresponding programs from the individual storage circuits.

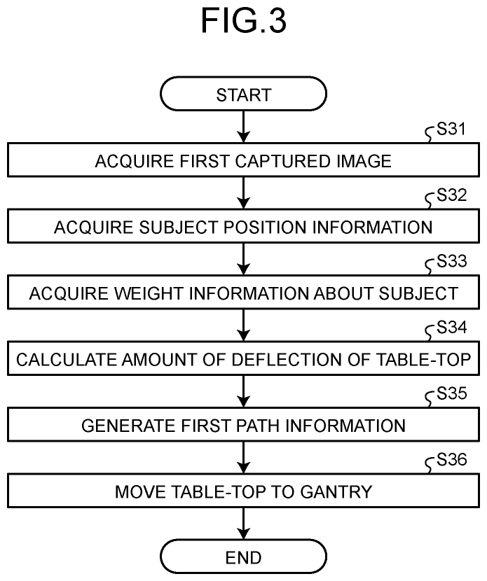

Explained below is a process performed by the magnetic resonance imaging apparatus 100 according to the first embodiment. FIG. 3 is a flowchart illustrating an example of the process executed by the magnetic resonance imaging apparatus 100 according to the first embodiment. Note that, in FIG. 3, the magnetic resonance imaging apparatus 100 starts the process after the determining function 173 of the processing circuitry 17 determines a site of the subject P to be imaged and the camera 18 captures an image of the subject P placed on the table-top 101.

First, the image acquiring function 142 of the processing circuitry 14 acquires the first captured image that is captured by the camera 18 (step S31). Then, the position acquiring function 143 of the processing circuitry 14 acquires subject position information indicating position and posture of the subject P placed on the table-top 101 from the first captured image acquired by the image acquiring function 142 (step S32).

The weight acquiring function 144 of the processing circuitry 14 then acquires weight information indicating weight of the subject P (step S33). The calculating function 145 of the processing circuitry 14 then calculates an amount of deflection of the table-top 101 based on the subject position information acquired by the position acquiring function 143 and the weight information acquired by the weight acquiring function 144 (step S34).

The path generating function 146 of the processing circuitry 14 then generates first path information indicating a path of the table-top 101 based on the amount of deflection of the table-top 101 calculated by the calculating function 145, the first path information including at least height of the table 10 during movement of the table-top 101 from the table 10 to the gantry 9 (step S35).

The movement control function 141 of the processing circuitry 14 then operates the driving member 103 based on the first path information generated by the path generating function 146 to move the table-top 101 to the gantry 9 along the path defined by the first path information (step S36), and the process is thus completed.

As described above, the magnetic resonance imaging apparatus 100 according to the first embodiment acquires subject position information indicating position of the subject P on the table-top 101, acquires weight information indicating weight of the subject P, and calculates an amount of deflection occurred in the table-top 101 based on the subject position information and the weight information. The magnetic resonance imaging apparatus 100 then generates, based on the amount of deflection of the table-top 101, first path information indicating a path along which the table-top 101 is moved from the table 10 to the gantry 9 where imaging of the subject P is performed, operates the moving mechanism that is capable of moving the table-top 101 from the table 10 to the gantry 9 to move the table-top 101 to the gantry 9 along the path indicated by the first path information.

With this configuration, the table-top 101 moves to the gantry 9 in accordance with the first path information, which achieves smooth movement to the gantry 9 even when there is a gap between the gantry 9 and the table 10. Therefore, the magnetic resonance imaging apparatus 100 and the table 10 can reduce rattling during movement of the table-top 101 to the gantry 9, which makes it possible to mitigate subject P's discomfort resulting from the rattling.

Note that the above-mentioned embodiment can also be implemented with appropriate variations by modifying a part of configuration or function that each device has. Explained below are some modifications according to the above-mentioned embodiment as other embodiments. Note that, there are explained below points that differ from the above-mentioned embodiment in main, and points in common with the contents already described are given the same symbols or numerals and their detailed explanation is not repeated. Moreover, the other embodiments described below may be implemented individually or in combination as appropriate.

### Second Embodiment

It has been explained, in the above-mentioned embodiment, to calculate an amount of deflection of the table-top 101 based on the subject position information indicating position of the subject P on the table-top 101 and the weight information indicating weight of the subject P. However, how to calculate an amount of deflection of the table-top 101 is not limited thereto. Explained below is another example of calculating an amount of deflection of the table-top 101 as a second embodiment.

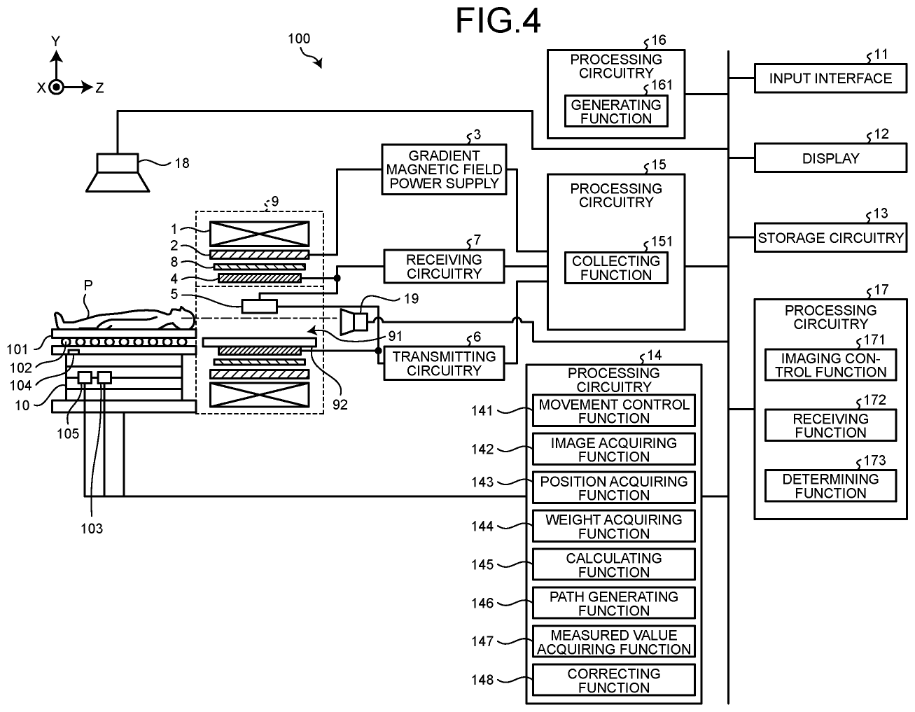

First, the magnetic resonance imaging apparatus 100 according to the second embodiment will be described below. FIG. 4 is a block diagram illustrating an example of the configuration of the magnetic resonance imaging apparatus 100 according to the second embodiment. The magnetic resonance imaging apparatus 100 according to the second embodiment further includes a camera 19.

Similar to the camera 18, the camera 19 is formed with, for example, a CCD image sensor or a CMOS image sensor. The camera 19 images the table-top 101 from a different position or angle than the camera 18. For example, as illustrated in FIG. 4, the camera 19 images the table-top 101 from the Z-axis direction through the bore 91 of the gantry 9.

Herein, the camera 19 is provided to image a state of deflection occurred in the table-top 101 (and the gantry 9) due to the placement of the subject P. The camera 19 generates an optical image of the table-top 101 (hereinafter referred to as “second captured image”) and transmits the second captured image to the processing circuitry 14.

Although the installation position of the camera 19 is not limited to the example as illustrated in FIG. 4, it is preferable to provide the camera 19 at a position able to image the table-top 101 before the table-top 101 is inserted into the bore 91.

Moreover, the processing circuitry 14 of the magnetic resonance imaging apparatus 100 according to the second embodiment includes the movement control function 141, the image acquiring function 142, the position acquiring function 143, the weight acquiring function 144, the calculating function 145, the path generating function 146, a measured value acquiring function 147, and a correcting function 148.

Herein, the measured value acquiring function 147 is an example of a measured value acquiring unit. The measured value acquiring function 147 acquires an amount of deflection of the table-top 101 from the second captured image. The amount of deflection of the table-top 101 from the second captured image is an example of a measured value. Specifically, the measured value acquiring function 147 acquires the second captured image from the camera 19. The measured value acquiring function 147 then acquires a state of deflection occurred in the table-top 101 as a measured value based on the acquired second captured image.

The correcting function 148 is an example of a correcting unit. The correcting function 148 corrects the amount of deflection of the table-top 101 calculated by the calculating function 145. Specifically, the correcting function 148 corrects the amount of deflection of the table-top 101 calculated by the calculating function 145 based on the measured value (amount of deflection) acquired by the measured value acquiring function 147.

For example, the correcting function 148 corrects the amount of deflection of the table-top 101 calculated by the calculating function 145 based on an amount of deflection at the end of the table-top 101 (and the table 10) acquired by the measured value acquiring function 147 before the table-top 101 is moved in the Z-axis direction.

Moreover, for example, the correcting function 148 corrects the amount of deflection of the table-top 101 calculated by the calculating function 145 based on an amount deflection at the end of the table-top 101 (and the table 10) acquired by the measured value acquiring function 147 after the table-top 101 starts moving in the Z axis direction.

Furthermore, the correcting function 148 corrects the amount of deflection of the table-top 101 calculated by the calculating function 145 based on an amount of deflection at the end of the table-top 101 (and the table 10) acquired by the measured value acquiring function 147 after the table-top 101 starts moving in the Z-axis direction and lands on the gantry 9.

The path generating function 146 generates first path information indicating a path along which the table-top 101 is moved from the table 10 to the gantry 9 based on the amount of deflection of the table-top 101 corrected by the correcting function 148. Note that the first path information generated by the path generating function 146 is the same as the above-mentioned first path information in the first embodiment.

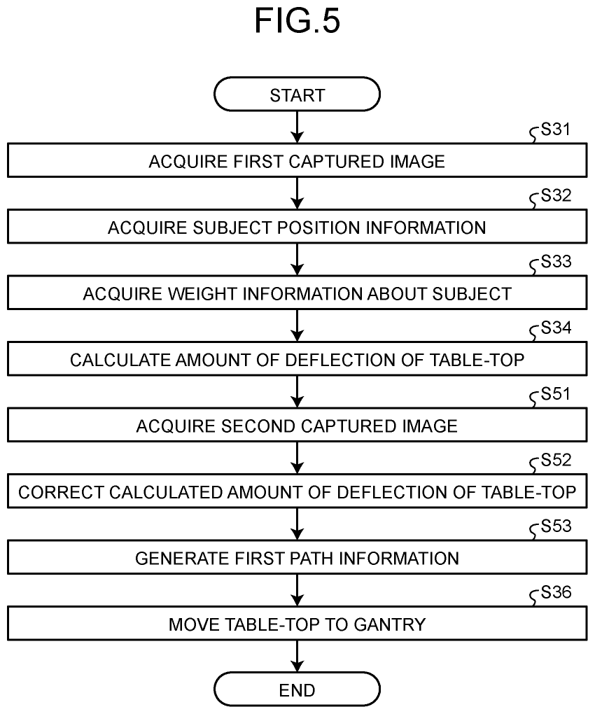

Explained below is a process performed by the magnetic resonance imaging apparatus 100 according to the second embodiment. FIG. 5 is a flowchart illustrating an example of the process performed by the magnetic resonance imaging apparatus 100 according to the second embodiment. Note that, as illustrated in FIG. 5, the magnetic resonance imaging apparatus 100 starts the process after the determining function 173 of the processing circuitry 17 determines a site of the subject P to be imaged and the camera 18 images the subject P placed on the table-top 101. Moreover, no explanation is made about steps shared with the process performed by the magnetic resonance imaging apparatus 100 according to the first embodiment.

The measured value acquiring function 147 of the processing circuitry 14 acquires the second captured image that is captured by the camera 19 and acquires a measured amount of deflection of the table-top 101 in the Y axis direction from the acquired second captured image (step S51). The correcting function 148 of the processing circuitry 14 then corrects the amount of deflection of the table-top 101 calculated by the calculating function 145 based on the measured amount of deflection acquired by the measured value acquiring function 147 (step S52).

The path generating function 146 then generates first path information indicating a path along which the table-top 101 is moved from the table 10 to the gantry 9 based on the amount of deflection of the table-top 101 corrected by the correcting function 148 (step S53).

As described above, the magnetic resonance imaging apparatus 100 according to the second embodiment acquires a second captured image including the table-top 101 on which the subject P is placed, and acquires, from the second captured image, a measured amount of deflection of the table-top 101 in the Y-axis direction. The magnetic resonance imaging apparatus 100 then corrects the calculated amount of deflection of the table-top 101 based on the measured amount of deflection of the table-top 101, calculates a height of the table 10 based on the corrected amount of deflection of the table-top 101, and generates first path information indicating a path along which the table-top 101 is moved from the table 10 to the gantry 9.

Because the table-top 101 moves to the gantry 9 in accordance with the first path information, smooth movement to the gantry 9 can be achieved even when there is a gap between the gantry 9 and the table 10, for example. Therefore, the magnetic resonance imaging apparatus 100 and the table 10 can reduce rattling during movement of the table-top 101 to the gantry 9, which makes is possible to mitigate subject P's discomfort resulting from the rattling.

### Third Embodiment

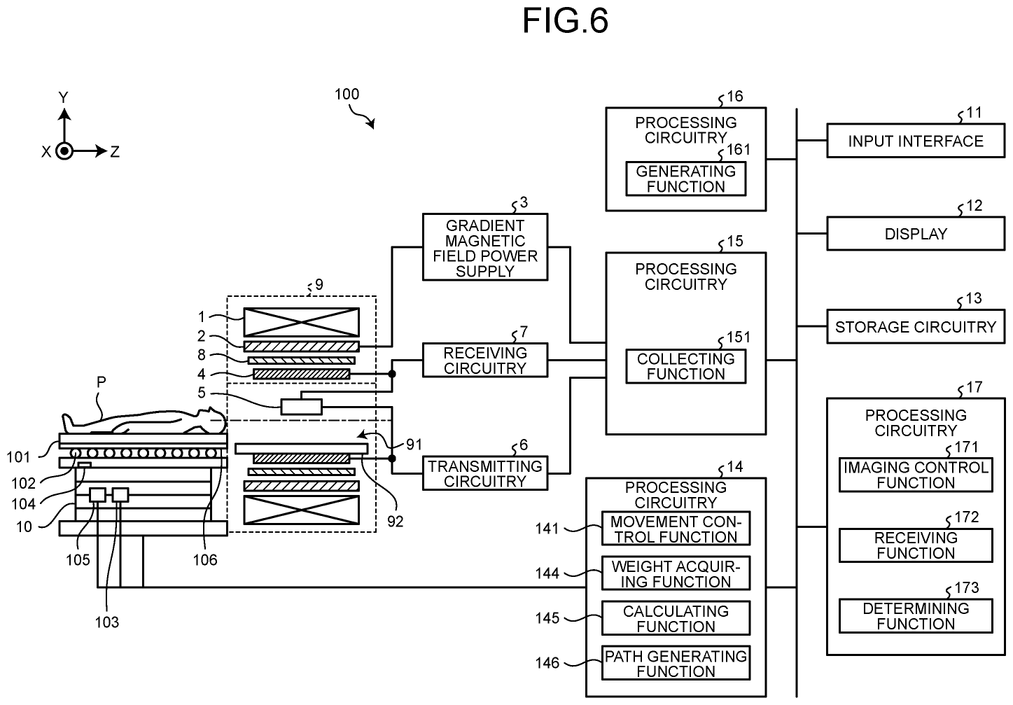

Explained below is the magnetic resonance imaging apparatus 100 according to a third embodiment. FIG. 6 is a block diagram illustrating an example of the configuration of the magnetic resonance imaging apparatus 100 according to the third embodiment. The table 10 of the magnetic resonance imaging apparatus 100 according to the third embodiment includes a weight sensor 106 below the table-top 101. Unlike the magnetic resonance imaging apparatus 100 according to the first and second embodiments, the magnetic resonance imaging apparatus 100 according to the third embodiment does not include the camera 18.

The weight sensor 106 is a sensor that detects weight of the subject P placed on the table-top 101. The weight sensor 106 is an example of a weight detecting unit. The weight sensor 106 detects a load applied to a surface of contact between the subject P and the table-top 101. The weight sensor 106 transmits the detected weight of the subject P to the processing circuitry 14.

The processing circuitry 14 of the magnetic resonance imaging apparatus 100 according to the third embodiment includes the movement control function 141, the weight acquiring function 144, the calculating function 145, and the path generating function 146.

The weight acquiring function 144 acquires load information. Specifically, the weight acquiring function 144 acquires load information indicating a position of the surface of contact between the subject P and the table-top 101 and a weight (load) applied to the position of the surface of contact between the subject P and the table-top 101, those being detected by the weight sensor 106.

The calculating function 145 calculates an amount of deflection of the table-top 101. Specifically, the calculating function 145 calculates an amount of deflection occurred in the table-top 101 due to the placement of the subject P based on the load information acquired by the weight acquiring function 144.

The path generating function 146 calculates a height of the table 10 based on the amount of deflection of the table-top 101 calculated by the calculating function 145 and generates first path information indicating a path along which the table-top 101 is moved from the table 10 to the gantry 9.

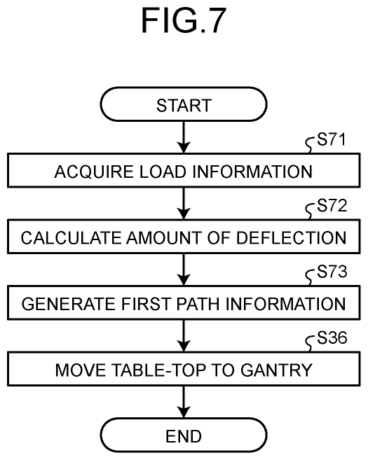

Explained below is a process performed by the magnetic resonance imaging apparatus 100 according to the third embodiment. FIG. 7 is a flowchart illustrating an example of the process performed by the magnetic resonance imaging apparatus 100 according to the third embodiment.

Note that, as illustrated in FIG. 7, the magnetic resonance imaging apparatus 100 starts the process after the determining function 173 of the processing circuitry 17 determines a site of the subject P to be imaged, and the weight sensor 106 detects a load applied to the surface of contact between the subject P and the table-top 101. Moreover, no explanation is made about steps shared with the process performed by the magnetic resonance imaging apparatus 100 according to the first embodiment.

First, the weight acquiring function 144 of the processing circuitry 14 acquires load information indicating a position of the surface of contact between the subject P and the table-top 101 and a load applied to the surface of contact between the subject P and the table-top 101, those detected by the weight sensor 106 (step S71). The calculating function 145 of the processing circuitry 14 then calculates an amount of deflection occurred in the table-top 101 due to the placement of the subject P based on the load information acquired by the weight acquiring function 144 (step S72).

The path generating function 146 then generates first path information indicating a path along which the table-top 101 is moved from the table 10 to the gantry 9 based on the amount of deflection of the table-top 101 calculated by the calculating function 145 (step S73).

As described above, the magnetic resonance imaging apparatus 100 according to the third embodiment acquires load information indicating a position of the surface of contact between the subject P and the table-top 101 and a load applied to the position of the surface of contact between the subject P and the table-top 101, and calculates an amount of deflection of the table-top 101 in the Y axis direction based on the load information. The magnetic resonance imaging apparatus 100 then generates first path information indicating a path along which the table-top 101 is moved from the table 10 to the gantry 9 based on the calculated amount of deflection of the table-top 101.

Because the table-top 101 moves to the gantry 9 in accordance with the first path information, smooth movement to the gantry 9 can be achieved even when there is a gap between the gantry 9 and the table 10, for example. Therefore, the magnetic resonance imaging apparatus 100 and the table 10 can reduce rattling during movement of the table-top 101 to the gantry 9, which makes it possible to mitigate subject P's discomfort resulting from the rattling.

### Fourth Embodiment

It has been explained in the above-mentioned embodiments that the table-top 101 on the table 10 moves to the gantry 9. It is explained in a fourth embodiment that the table-top 101 on the gantry rail 92 moves to the table 10.

When, for example, the table-top 101 is inside the gantry 9, because the table 10 releases from the load by the table-top 101 on which the subject P is placed, a phenomenon occurs in which the deflection of the table 10 disappears and the height of the table 10 increases.

Therefore, when the table-top 101 on the gantry rail 92 is moved to the table 10, the magnetic resonance imaging apparatus 100 according to the fourth embodiment operates the moving mechanism to move the table-top 101 to the table 10 while decreasing the height of the table 10 to prevent rattling in the table-top 101, thereby reducing the rattling in the table-top 101.

Specifically, the path generating function 146 of the processing circuitry 14 generates second path information indicating a path along which the table-top 101 is moved from the gantry 9 to the table 10 based on an amount of deflection of the table-top 101. The second path information includes at least a vertical height of the table-top 101 at each position through which the table-top 101 is moved from the gantry 9 to the table 10. The second path information also includes the height of the table 10 corresponding to the moving position of the table-top 101.

Note that, for position detection of the table-top 101 inside the gantry 9, as an example, a table-top position detection sensor (not illustrated) is provided inside the gantry 9. For example, the table-top position detection sensor uses a position of the table-top 101 in an initial state before the table-top 101 is moved into the table 10 as a reference position, and detects a distance by which the table-top 101 has moved in the Z axis direction from the reference position as a table-top position. Moreover, the table-top position detection sensor transmits the detected table-top position to the processing circuitry 14.

For example, the height of the table 10 included in the second path information is a height of the table 10 before the table-top 101 moves from the gantry 9, a height of the table 10 immediately after the table-top 101 moves from the gantry 9, and a height of the table 10 after the table-top 101 moves from the gantry 9.

The movement control function 141 of the processing circuitry 14 operates the moving mechanism to move the table-top 101 to the table 10 along the path indicated by the second path information. Note that the moving mechanism is provided inside the gantry rail 92. The moving mechanism is capable of moving the table-top 101 in the horizontal direction and the vertical direction.

As described above, the magnetic resonance imaging apparatus 100 according to the fourth embodiment generates second path information indicating a path along which the table-top 101 is moved from the gantry 9 to the table 10 based on an amount of deflection of the table-top 101, and operates the moving mechanism based on the second path information to move the table-top 101 on which the subject P is placed from the gantry 9 to the table 10.

For example, when the table-top 101 is moved from the gantry 9, the table height decreases to prevent rattling, which makes it possible to move the table-top 101 to the table 10 without rattling.

### Fifth Embodiment

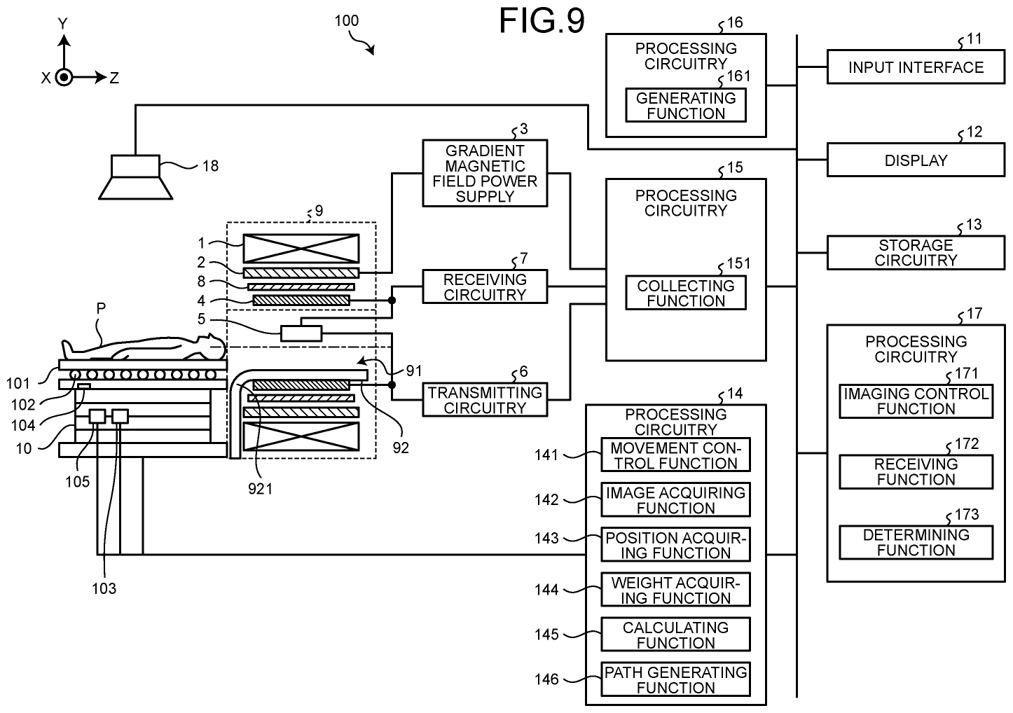

It has been explained, in the above-mentioned embodiments, that the longitudinal direction of the gantry rail 92 provided on the gantry 9 is parallel to the table 10. It is explained, in the fifth embodiment, that the gantry rail 92 bends at an end closer to the table with reference to FIG. 9.

As illustrated in FIG. 9, the end of the gantry rail 92 closer to the table 10 further extends toward a lower part of the gantry 9. Moreover, a corner (hereinafter also referred to as “bend 921”) of the further-extended part of the gantry rail 92 is curved.

Even if the moving member 102 of the table-top 101 is in contact with the gantry rail 92 when the table-top 101 is moved into the gantry 9, presence of the bend 921 in the gantry rail 92 can reduce an impact of the moving member 102 riding on the gantry rail 92 and achieve soft landing on the gantry rail 92.

Note that, if the configuration of the present embodiment is adopted, the path generating function 146 generates first path information indicating a path along which the table-top 101 is moved from the table 10 to the gantry 9 with position or shape of the bend 921 provided on the gantry rail 92 being further taken into consideration.

For example, the path generating function 146 generates, as a path before the table-top 101 is moved into the Z-axis direction, first start-up information based on an amount of deflection at the end of the table-top 101 (and the table 10) to instruct movement of the table 10 to a position lower than the bend 921 of the gantry 9.

Moreover, as a path after the table-top 101 starts moving in the Z-axis direction, the path generating function 146 generates first start-up information based on an amount of deflection at the end of the table-top 101 (and the table 10) to instruct increase in height of the table 10 to the same level as the gantry rail 92 so that the moving member 102 of the table-top 101 rides on the bend 921 of the gantry 9.

Furthermore, as a path after the table-top 101 starts moving in the Z-axis direction and lands on the gantry 9, the path generating function 146 generates first start-up information based on an amount of deflection at the end of the table-top 101 (and the table 10) to instruct to make the height of the table 10 and the height of the gantry rail 92 equal so that the moving member 102 of the table-top 101 rides on the gantry rail 92 of the gantry 9. Note that, as for the height of the table 10, gradual movement is preferable so that the moving member 102 of the table-top 101 softly lands on the gantry rail 92 of the gantry 9.

For example, if the gantry 9 has the bend 921, control of the moving position of the table-top 101 is performed while the table height increases for purpose of an improvement in throughput so that the table-top 101 is moved without rattling at the bend 921. This makes it possible to reduce rattling in the table-top 101 on which the subject P is placed. Since no rattling occurs in the table-top 101, the table 10 can mitigate subject P's discomfort resulting from the movement of the table-top 101.

The examples have been explained in the respective embodiments above in which a “processor” reads a program corresponding to each processing function from storage circuitry and excuses the program; however, the embodiments are not limited thereto. The term “processor” means circuitry, for example, a central processing unit (CPU), a graphics processing unit (GPU), an application specific integrated circuit (ASIC), a programmable logic device (e.g., simple programmable logic device (SPLD), complex programmable logic devices (CPLD), and field programmable gate array (FPGA)), etc.

If the processor is a CPU, for example, the processor reads and executes a program stored in the storage circuitry to realize each processing function. If the processor is an ASIC, instead of storing the program in the storage circuitry, the processing function is directly incorporated into a circuit of the processor as a logic circuit. Note that each processor of the present embodiments is not limited to form as a single circuit. One processor may be formed by a combination of two or more independent circuits and realize its processing function. Moreover, two or more components as illustrated in FIG. 1 may be integrated into one processor and realize its processing function.

Herein, a program executed by the processor is provided in such a manner that the program is pre-embedded on a read only memory (ROM), storage circuitry, etc. The program may be provided as a file in a format installable in or executable by these devices in such a manner that the program is stored in a computer-readable storage medium, such as a compact disc-read only memory (CD-ROM), a flexible disk (FD), a compact disc-recordable (CD-R), a digital versatile disc (DVD), etc.

Moreover, it is allowable to store the program in a computer connected to a network, such as the Internet, and provide or distribute the program by download via the network. For example, the program is configured with modules containing the respective functional units as described above. In terms of actual hardware, a CPU reads the program from a storage medium such as a ROM and executes the program; thereby, the modules are loaded onto the main memory and generated on the main memory.

With at least one of the embodiments described above, it is possible to reduce rattling in a table-top on which a subject is placed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A magnetic resonance imaging apparatus comprising:
a table apparatus including a table-top on which a subject is placed; and
a processor configured to
acquire subject position information indicating a position of the subject on the table-top,
acquire weight information indicating a weight of the subject,
calculate an amount of deflection occurred in the table-top based on the subject position information and the weight information,
generate, based on the amount of deflection of the table-top, first path information indicating a path along which the table-top is moved from the table apparatus to a gantry where imaging of the subject is performed, and
operate a moving mechanism capable of moving the table-top from the table apparatus to the gantry to move the table-top from the table apparatus to the gantry along the path indicated by the first path information.

2. The magnetic resonance imaging apparatus according to claim 1, wherein the processor is configured to acquire the subject position information based on a first captured image that is an image of an upper surface of the table-top.

3. The magnetic resonance imaging apparatus according to claim 1, wherein the processor is configured to
acquire a state of deflection occurred in the table-top as a measured value based on a second captured image that is an image of the table-top,
correct the calculated amount of deflection of the table-top based on the measured value, and
generate the first path information based on the corrected amount of deflection of the table-top.

4. The magnetic resonance imaging apparatus according to claim 1,
wherein the processor is configured to generate second path information indicating a path along which the table-top is moved from the gantry to the table apparatus based on the amount of deflection of the table-top, and
operate a moving mechanism capable of moving the table-top from the gantry to the table apparatus to move the table-top to the table apparatus along the path indicated by the second path information.

5. The magnetic resonance imaging apparatus according to claim 1, wherein the processor is configured to generate the first path information including at least a vertical height of the table-top at each position through which the table-top is moved from the table apparatus to the gantry.

6. The magnetic resonance imaging apparatus according to claim 5, wherein
the table apparatus includes a moving mechanism configured to move the table-top in a horizontal direction and a vertical direction, and
the processor is configured to operate the moving mechanism based on the first path information to move the table-top to the gantry along the path indicated by the first path information.

7. The magnetic resonance imaging apparatus according to claim 4, wherein the processor is configured to generate the second path information including at least a vertical height of the table-top at each position through which the table-top is moved from the gantry to the table apparatus.

8. The magnetic resonance imaging apparatus according to claim 7, wherein
the table apparatus includes a moving mechanism configured to move the table-top in a horizontal direction and a vertical direction, and
the processor is configured to operate the moving mechanism based on the second path information to move the table-top from the gantry to the table apparatus along the path indicated by the second path information.

9. A table apparatus comprising:
a table-top on which a subject is placed; and
a processor configured to
acquire subject position information indicating a position of the subject on the table-top,
acquire weight information indicating a weight of the subject,
calculate an amount of deflection occurred in the table-top based on the subject position information and the weight information,
generate, based on the amount of deflection of the table-top, first path information indicating a path along which the table-top is moved to the gantry where imaging of the subject is performed, and
operate a moving mechanism capable of moving the table-top to the gantry to move the table-top to the gantry along the path indicated by the first path information.
